SEQUENCE LISTING (18 sequences)

SEQ ID NO: 1 - DNA, 48 bp, Artificial Sequence
  Synthetic
ggtttgtctggtcaaccaccgcggtctcagtggtgtacggtacaaacc

SEQ ID NO: 2 - DNA, 38 bp, Artificial Sequence
  Synthetic
ggcttgtcgacgacggcggtctccgtcgtcaggatcat

SEQ ID NO: 3 - DNA, 200 bp, Artificial Sequence
  Synthetic
gaggaccgccctgggcctgggagaatcccttccccctcttccctcgtgatctgcaactccagtctttctagaagatggtttgtctggtcaaccaccgcggtctcagtggtgtacggtacaaaccgggcgggagtcttctgggcaggcttaaaggctaacctggtgtgtgggcgttgtcctgcaggggaattgaacaggtg

SEQ ID NO: 4 - DNA, 76 bp, Artificial Sequence
  Synthetic
gaggaccgccctgggcctgggagaatcccttccccctcttccctcgtgatctgcaactccagtctttctagaagat

SEQ ID NO: 5 - DNA, 76 bp, Artificial Sequence
  Synthetic
gggcgggagtcttctgggcaggcttaaaggctaacctggtgtgtgggcgttgtcctgcaggggaattgaacaggtg

SEQ ID NO: 6 - RNA, 18 nt, Artificial Sequence
  Synthetic
gucuuucuagaagauggg

SEQ ID NO: 7 - DNA, 48 bp, Artificial Sequence
  Synthetic
ggtttgtctggtcaaccaccgcggactcagtggtgtacggtacaaacc

SEQ ID NO: 8 - DNA, 38 bp, Artificial Sequence
  Synthetic
ggcttgtcgacgacggcggactccgtcgtcaggatcat

SEQ ID NO: 9 - DNA, 131 bp, Artificial Sequence
  Synthetic
gcaaaactacaggttattattgcttgtgatccgcctcggagtattttccatcgggtttgtctggtcaaccaccgcggactcagtggtgtacggtacaaaccaggtagattaaagacatgctcacccgagtt

SEQ ID NO: 10 - RNA, 18 nt, Artificial Sequence
  Synthetic
gucuuuaaucuaccucga

SEQ ID NO: 11 - DNA, 48 bp, Artificial Sequence; identical to SEQ ID NO: 1
  Synthetic

SEQ ID NO: 12 - DNA, 48 bp, Artificial Sequence
  Synthetic
ccaaacagaccagttggtggcgccagagtcaccacatgccatgtttgg

SEQ ID NO: 13 - DNA, 38 bp, Artificial Sequence; identical to SEQ ID NO: 2
  Synthetic

SEQ ID NO: 14 - DNA, 38 bp, Artificial Sequence
  Synthetic
ccgaacagctgctgccgccagaggcagcagtcctagta

SEQ ID NO: 15 - DNA, 43 bp, Artificial Sequence
  Synthetic
ggtttgtctggtcaaccaccgcggtctccgtcgtcaggatcat

SEQ ID NO: 16 - DNA, 43 bp, Artificial Sequence
  Synthetic
ccaaacagaccagttggtggcgccagaggcagcagtcctagta

SEQ ID NO: 17 - DNA, 43 bp, Artificial Sequence
  Synthetic
ggcttgtcgacgacggcggtctcagtggtgtacggtacaaacc

SEQ ID NO: 18 - DNA, 43 bp, Artificial Sequence
  Synthetic
ccgaacagctgctgccgccagagtcaccacatgccatgtttgg

(19) United States
(12) Patent Application Publication
(10) Pub. No.: US 2022/0369610 A1
(43) Pub. Date: Nov. 24, 2022

(54) HIGH FREQUENCY TARGETED ANIMAL TRANSGENESIS

(71) Applicant: The Jackson Laboratory, Bar Harbor, ME (US)

(72) Inventors: Michael V. Wiles, Bar Harbor, ME (US); Benjamin E. Low, Bar Harbor, ME (US)

(73) Assignee: The Jackson Laboratory, Bar Harbor, ME (US)

(21) Appl. No.: 17/767,311

(22) PCT Filed: Oct. 8, 2020
(86) PCT No.: PCT/US2020/054745

§ 371 Date: Apr. 7, 2022

Related U.S. Application Data
(60) Provisional application No. 62/913,092, filed on Oct. 9, 2019.

(51) Int. Cl. A01K 67/027; C12N 15/90

(52) U.S. Cl. CPC A01K 67/0278; C12N 15/907; A01K 2217/072; A01K 2227/105; C12N 2310/20; A01K 2207/15

(57) ABSTRACT

The present disclosure provides methods and compositions for high frequency mouse transgenesis using, for example, a Bxb1 landing pad.

### RELATED APPLICATION

This application claims the benefit under 35 U.S.C. § 119(e) of U.S. provisional application No. 62/913,092, filed Oct. 9, 2019, which is incorporated by reference herein in its entirety.

### FEDERALLY SPONSORED RESEARCH

The invention was made with government support under R24 OD016473 and R21 OD023800 awarded by National Institutes of Health. The government has certain rights in the invention.

### BACKGROUND

The genome engineering revolution continues to drive rapid modification of the mouse genome, creating more elegantly complex strains of mutant mice faster than ever before. While small genome modifications are simple and efficient, the reliance on homologous recombination for precise insertion of large donor DNA remains problematic. Generating humanized mice requires incorporating control regions of a gene in order to recapitulate the intended expression pattern and function. This is one reason for the use of random and inherently shambolic transgenesis, which can suffer from low efficiency, partial/incomplete integration, and multicopy concatemerization. Such insertions often deposit into active loci, resulting in deleterious positional effects on transgene expression and the unintentionally disrupted endogenous gene(s). Consequently, large transgenic projects often require substantial characterization time and extra rounds of breeding.

### SUMMARY

The present disclosure demonstrates that large, single copy insertions of exogenous DNA into the mouse genome is achievable using the Bxb1 integrase system. Thus, provided herein, in some aspects, is a system that utilizes a combination of gene editing tools (e.g., CRISPR/Cas9) and a Bxb1 integrase to insert single copies of large transgenes into a specific locus. In the presence of Bxb1 integrase, an attP site is recombined with an attB site to convert the sites into an attR (“Right”) and attL (“Left”) site (FIG. 1). This system, in some embodiments, excludes the plasmid/bacterial donor DNA vector sequence integration into the genome, which has been shown to result in transgene silencing. Herein, 30.6 kilobases (kb) of human DNA was integrated into the C57BL/6J mouse Rosa26 locus with 11% efficiency ( 4/35). Surprisingly, all four independent lines have demonstrated germline transmission of the transgenic allele, free from off-target contamination, within 3-6 months from the microinjection date. Further, the gene is transcribed appropriately in liver. These results demonstrate the ability of this system to deliver intact, single-copy transgenes into a defined locus in a short period of time, providing precision transgenesis, a powerful tool for rapid and reliable genetic engineering of animal models.

The present disclosure provides, in some aspects, methods for targeted insertion of large transgenes (e.g., a length of at least 20 kilobases) in an animal (e.g., a mammal, such as a rodent, for example, a mouse) genome, with limited off-target modification of the genome. Surprisingly, mice genetically engineered to harbor a Bxb1 att (attachment site) landing pad enable the insertion of large transgenes at high insertion frequencies. Further, the Bxb1 system is not known to exhibit pseudo integration sites in the mouse genome, in particular (see, e.g., Russell J P et al. BioTechniques 2006; 40:460-464).

In some embodiments, the Bxb1 landing pad mouse strains of the present disclosure are generated directly in mice through pronucleus microinjection of CRISPR/Cas9 gene editing tools that include polynucleotides encoding Cas9 nuclease (or a variant or homolog thereof), a guide RNA (gRNA) targeting a genomic locus, such as a safe harbor locus (e.g., Rosa26 or Hip11 locus), and a (at least one) single-stranded DNA (ssDNA) containing the Bxb1 attachment site(s) flanked by homology arms to the safe harbor locus. Use of these CRISPR/Cas9 gene editing tools, for example, produces a mouse line/strain with little to no off-target changes (outside of the intended genomic locus) in the genome. The established Bxb1 landing pad mouse lines may then be used as a platform for the insertion and subsequent analyses of transgenes of interest. For example, a donor DNA that includes a transgene of interest and corresponding (cognate) Bxb1 attachment site(s) may be microinjected with Bxb1 integrase (or polynucleotide encoding Bxb1 integrase) to produce a transgenic mouse harboring the genomically-integrated transgene of interest.

Some aspects of the present disclosure provide mammals (or other animals) comprising within its genome (e.g., a genomic locus) a first Bxb1 attachment site (e.g., attP or attB) and a second Bxb1 attachment site (e.g., modified attP* or modified attB*). In some embodiments, the mammals further comprise a polynucleotide encoding a Bxb1 integrase. The polynucleotide may be, for example, flanked by the first and second Bxb1 attachment sites. Thus, the Bxb1 integrase may be genomically encoded.

Other aspects of the present disclosure provide mammalian embryos (or other animal embryos) comprising within its genome (e.g., a genomic locus) a first Bxb1 attachment site and a second Bxb1 attachment site. In some embodiments, the mammalian embryos further comprise a polynucleotide encoding a Bxb1 integrase, optionally wherein the polynucleotide is flanked by the first and second Bxb1 attachment sites.

In some embodiments, a first Bxb1 attachment site is selected from an attP site, a modified attP* site, an attB site, and a modified attB* site. In some embodiments, a second Bxb1 attachment site is selected from an attP site, a modified attP* site, an attB site, and a modified attB* site. In some embodiments, a first and second Bxb1 attachment sites are heterologous relative to each other.

In some embodiments, an attP site comprises the sequence of SEQ ID NO: 1. In some embodiments, a modified attP* site comprises the sequence of SEQ ID NO: 7. In some embodiments, an attB site comprises the sequence of SEQ ID NO: 2. In some embodiments, a the modified attB* site comprises the sequence of SEQ ID NO: 8.

In some embodiments, the first and second Bxb1 attachment sites are separated from each other by 50 to 500 nucleotide bases.

In some embodiments, the genomic locus is a safe harbor locus, optionally a Rosa26 locus. Other loci may be targeted.

In some embodiments, the mammal is a rodent, for example, a mouse or rat. In some embodiments, the mammalian embryo is a rodent embryo, for example, a mouse embryo or a rat embryo. Other mammals, and non-mammals, may be used.

Also provided herein are methods comprising introducing into a mammalian embryo (a) a donor polynucleotide comprising a sequence of interest flanked by a first cognate Bxb1 attachment site and a second cognate Bxb1 attachment site, and (b) a Bxb1 integrase or a polynucleotide encoding a Bxb1 integrase. Further provided herein are methods comprising introducing into the mammalian embryo a donor polynucleotide comprising a sequence of interest flanked by a first cognate Bxb1 attachment site and a second cognate Bxb1 attachment site.

In some embodiments, the methods further comprise implanting the mammalian embryo into a pseudopregnant female mammal. In some embodiments, the methods further comprise collecting from the female mammal a progeny mammal. In some embodiments, the method further comprises screening the progeny mammal for the presence or absence of the sequence of interest integrated into the genome of the progeny mammal.

In some embodiments, the donor polynucleotide, the Bxb1 integrase, and/or the polynucleotide encoding a Bxb1 integrase is/are introduced into the mammalian embryo via microinjection. Other transfection methods may be used.

In some embodiments, a sequence of interest comprises a gene of interest.

In some embodiments, a sequence of interest has a size of at least 10 kb, at least 15 kb, at least 20 kb, at least 25 kb, or at least 30 kb.

Further aspects of the present disclosure provide methods for producing a Bxb1 landing pad mammal, the method comprising: (a) introducing into a mammalian embryo (i) a Cas9 nuclease or a polynucleotide encoding a Cas9 nuclease, (ii) a first guide RNA (gRNA) or a polynucleotide encoding a gRNA that targets a first genomic site (e.g., locus) in the mammalian embryo, (iii) a first single-stranded DNA (ssDNA) donor comprising a first Bxb1 attachment site flanked by a left homology arm and a right homology arm; optionally (iv) a second guide RNA (gRNA) or a polynucleotide encoding a gRNA that targets a second genomic (e.g., locus) in the mammalian embryo, and (v) a second ssDNA comprising a second Bxb1 attachment site flanked by a left homology arm and a right homology arm; and (b) implanting the mammalian embryo cell into a pseudopregnant female mammal, wherein the pseudopregnant female mammal is capable of giving birth to a progeny mammal.

In some embodiments, a first ssDNA further comprises a second Bxb1 attachment site upstream or downstream from the first Bxb1 attachment site, wherein both the first and second Bxb1 attachment sites are flanked by the left homology arm and the right homology arm.

In some embodiments, a mammalian embryo comprises a polynucleotide encoding a Bxb1 integrase, or the method further comprises introducing into the mammalian embryo a polynucleotide encoding a Bxb1 integrase.

In some embodiments, the methods further comprise collecting the progeny mammal.

Additional aspects of the present disclosure provide mammals (e.g., rodents, such as mice or rats) comprising a mammalian embryo described herein.

Other aspects of the present disclosure provide mammal or mammalian embryos comprising within its genome (e.g., a genomic locus) a single Bxb1 attachment site, as well as methods of producing and using the mammals or mammalian embryos.

### BRIEF DESCRIPTION OF THE DRAWINGS

FIGS. 1A-1C: Bxb1 integrase utilizes an attachment site to integrate exogenous DNA (e.g., attP selectively placed in the genome along with its reciprocal attB site in a vector). In a first version (FIG. 1A), a single Bxb1 attachment site in the genome serves to integrate the entire donor vector into the genome. To minimize insertion of undesired plasmid backbone, the vector was first processed into a minicircle. Letters (ABC . . . ) are shown to demonstrate sequence orientation. In a second version (FIG. 1B), two Bxb1 attachment sites (identical except for the dinucleotide bases that confer specificity) were utilized. With this strategy, it is not necessary to convert the donor plasmid into minicircles prior to delivery. Rather, recombination between the dual heterologous attachment sites (engineered/modified e.g., using alternative dinucleotide base pairs) functions to exclude the plasmid backbone from integration into the landing pad. In a third version (FIG. 1C), the zygote is “primed” with transgenic Bxb1 protein to facilitate efficient recombination. Flanked by two Bxb1 attachment sites, this Bxb1 integrase transgene is eliminated following successful Bxb1-mediated replacement with the donor sequence.

FIGS. 2A-2B: (FIG. 2A) Bxb1 attachment site sequences shown with the canonical dinucleotide (GT) 3′ overhang. Sequences are SEQ ID NOS: 11-18, top to bottom. (FIG. 2B) Non-limiting list of the alternative dinucleotide options that enable greater design flexibility and sequential modification. Asterisks indicate palindromic overhangs (*), identical double base overhangs (**), and the canonical overhang (***).

FIG. 3: Summary of screening strategy. Overview of simple PCR-based screening strategy for identifying and confirming sequence of the recombinant allele, as well as detection of an off-target integration event outside of the landing pad.

FIG. 4: Long-Range PCR to confirm knock-in. Example of the use of long-range PCR assays to verify the recombinant allele is correct and intact. Unlike random transgenesis where the flanking regions are unknown, or homologous recombination where long homology arms can make this type of assay difficult to impossible, candidate alleles generated using our system allows for long-range PCR verification using an “In/Out” PCR strategy. In this example, nearly the entire 30.6 kb insert is captured by just two PCRs, each with one primer binding to a site in the transgene (“In”) and the other binding to the genome adjacent to the landing pad (“Out”).

FIG. 5: RT-PCR to confirm transcript. Evidence that the transgene is expressed as intended was generated using mRNA extracted from the liver, in this case the expected tissue for expression of the human transgene, converted to cDNA, amplified and sequence verified. Schematic showing alignment with the reference across the splice sites is shown, along with agarose gel electrophoresis of the amplicon from three independent founder lines demonstrating that the human-derived transcript was expressed and splice appropriately.

FIG. 6: Efficiency of plasmid integration into a Bxb1 mouse with one landing pad (Bxb1v1) versus two landing pads (Bxb1v2). Summary of all experiments to date using Bxb1 Integrase mRNA under a multitude of test conditions. For three of the four version 1 landing pad strains (single attP site), successful recombinant alleles were generated. Both of the version 2 (dual attP site) landing pad mouse strains successfully generated recombinant alleles. In the background where the most experiments have been performed (B6), the dual site (version 2) system appears to be approximately 3-4 times more efficient than the single site version (version 1).

### DETAILED DESCRIPTION

Historically, the introduction of large (>10 kilobases) transgenes in mice has been accomplished by either embryonic stem cell manipulation or more commonly by random transgenesis (and only very occasionally by CRISPR-mediated HDR) directly in the zygote. Targeted transgenesis typically relies on the use of extensive homology arms flanking the donor transgene, resulting in even larger vector sizes. The use of such methods presents technical challenges in production and handling and requires extensive downstream work to mitigate any unintended consequences. Animal (e.g., mammal, such as rodent, for example, mouse) models created using random transgenesis often suffer from positional effects, for example, disruption of native genes at the sites of integration/s, and aberrant transgene expression levels. Multicopy concatemers can lead to vast overexpression or with multiple insertions scattered over the genome leading to segregation of the transgenes during breeding, with subsequent expression changes and instability of the required phenotype. Further, the coincident inclusion of elements from the plasmid backbone can result in transgene silencing, nullifying the utility of a potential mammalian model.

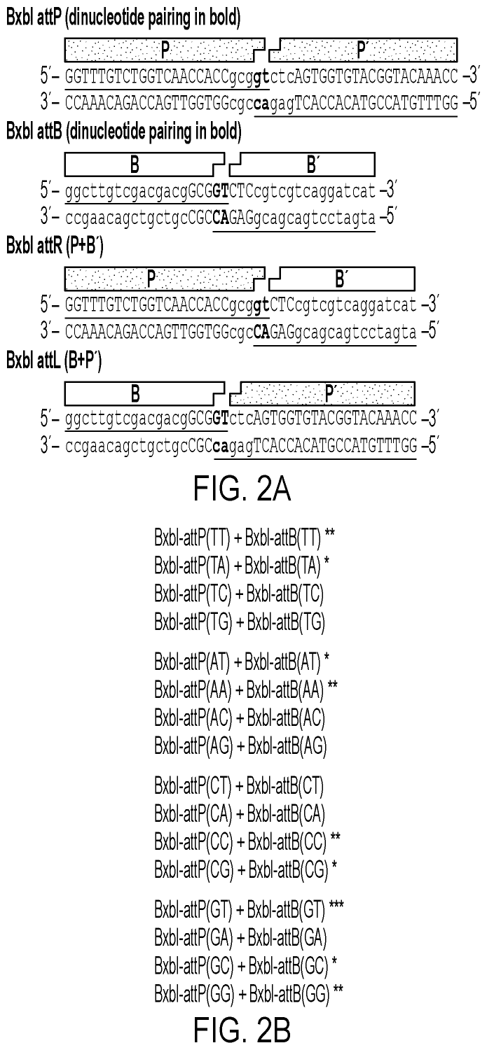

The serine recombinase encoded by the Bxb1 mycobacteriophage offers a solution to many of the challenges posed by traditional techniques, for example, large-scale humanization of the mouse genome. This serine recombinase may be used for the introduction of any human, mouse (or any other species), or synthetic construct to a mammalian genome. In nature, the Bxb1 integrase functions to perform DNA strand exchange between unique attachment sites in the phage (“attP”) and its bacterial host (“attB”) during its lysogenic phase. Depending on the relative orientation of the attP and attB sites, the reaction can result in excision, inversion or integration of sequences between the recognition sites, and is not reversible unless an additional protein, an excisionase, is present. Each attachment site is <50 nucleotide base pairs (bp) in length making it facile to use in molecular cloning as well as for insertion into host genomes using now common gene editing techniques. The Bxb1 integrase works in eukaryotic cells and does not require any additional host factors to function. Further, it has been shown to function at high efficiency in cells, is unidirectional and has no detectable pseudo sites in the mouse genome. The system also lends itself to enhancement, as the two central dinucleotides in the attachment sites are solely responsible for the specificity of the recombination event (see, e.g., FIG. 2B). These combined attributes render this system useful for directly modifying mammalian (e.g., mouse) zygotes.

The present disclosure describes how the Bxb1 integrase system was used to engineer mouse strains with a “landing pad” in a safe harbor locus. These strains enable, in some embodiments, the introduction of single-copy transgenes in a defined orientation without contaminating vector sequence in the recombinant allele. Knowledge of the precise position of the recombinant allele, combined with high efficiency, greatly assists in the identification of founders and subsequent verification of the transgene. It should be understood that while the present disclosure describes how the Bxb1 integrase system was used to engineer mouse strains, it may also be used to engineer other animals, for example, other mammals (e.g., non-human mammals) with a “landing pad.”

### Production of Bxb1 Landing Pad Strains

A Bxb1 landing pad animal is an animal that includes in its genome a (at least one) Bxb1 attachment site (e.g., an attB site, Bxb1 attP site, and/or modified versions thereof). In some embodiments, the animal genome comprises a Bxb1 attP site (SEQ ID NO: 1) or a modified Bxb1 attP* site (SEQ ID NO: 7). In some embodiments, the animal genome comprises a Bxb1 attB site (SEQ ID NO: 2) or a modified Bxb1 attB* site (SEQ ID NO: 8). Non-limiting examples of other dinucleotide-modified Bxb1 attachment sites are provided in FIG. 2B. The animal may be any animal, such as a lab animal or a livestock/farm animal. In some embodiments, the animal is a mammal. In some embodiments, the mammal is a rodent. The rodent may be, for example, a mouse or a rat. Other animals, such as poultry (e.g., chickens), are also contemplated herein.

The integrase encoded by the mycobacteriophage Bxb1 catalyzes strand exchange between attP and attB, the attachment sites for the phage and bacterial host, respectively. Although the DNA sites are relatively small (<50 bp), the reaction is highly selective for these sites and is also strongly directional (see, e.g., Singh A et al. PLoS Genetics 2013; 9(5): e1003490). The Bxb1 attB sites show at least seven unique and specific optimal variations, plus a further nine suboptimal variations in an internal dinucleotide recognition sequence, allowing the same Bxb1 recombinase enzyme to use a series of different constructs at the same time each with its specific dinucleotide address (see. e.g., Ghosh P et al. J. Mol Biol. 2006; 349:331-348). Thus, contemplated herein is the use of Bxb1 attP sites and modified attP* sites (e.g., modified relative to the sequence of SEQ ID NO: 1), as well as the use of Bxb1 attB sites and modified attB* sites (e.g., modified relative to the sequence of SEQ ID NO: 2)

It should be understood, unless noted otherwise, that the Bxb1 landing pad animal (e.g., a mammal, such as a rodent, for example, a mouse) strains may include a Bxb1 attP site, a modified Bxb1 attP site, a Bxb1 attB site, modified Bxb1 attB site, or any combination thereof. The corresponding donor polynucleotide to be inserted into the Bxb1 landing pad should include the cognate Bxb1 attachment site(s). Thus, if the Bxb1 landing pad animal strain includes a Bxb1 attP site, the corresponding polynucleotide (e.g., circular donor DNA) to be inserted into the Bxb1 landing pad should include a Bxb1 attB site; and if the Bxb1 landing pad animal strain includes a Bxb1 attB site, the corresponding polynucleotide to be inserted into the Bxb1 landing pad should include a Bxb1 attP site.

The Bxb1 attachment site(s), in some embodiments, is/are located in a safe harbor locus, which is an open chromatin region of a genome. Genomic safe harbors (GSHs) are sites in the genome able to accommodate the integration of new genetic material in a manner that ensures that the newly inserted genetic elements: (i) function predictably and (ii) do not cause alterations of the host genome posing a risk to the host cell or organism (see, e.g., Papapetrou EP and Schambach A Mol Ther 2016; 24(4): 678-684).

Non-limiting examples of safe harbor loci that may be used as provided herein include the Rosa26 locus, the Hip11 locus, the Hprt locus, and the Tigre locus. Thus, in some embodiments, the Rosa26 locus of a mouse (or other mammalian) strain of the present disclosure includes a Bxb1 attP site or a modified attP* site. In some embodiments, the Rosa26 locus includes a Bxb1 attB site or a modified Bxb1 attB* site.

In other embodiments, the Hip11 locus of a mouse (or other mammalian) strain of the present disclosure includes a Bxb1 attP site or a modified attP* site. In some embodiments, the Hip11 locus includes a Bxb1 attB site or a modified attB* site. In yet other embodiments, the Hprt locus of a mouse (or other mammalian) strain of the present disclosure includes a Bxb1 attP site or a modified attP* site. In some embodiments, the Hprt locus includes a Bxb1 attB site or a modified attB* site. In still other embodiments, the Tigre locus of a mouse (or other mammalian) strain of the present disclosure includes a Bxb1 attP site or a modified attP* site. In some embodiments, the Tigre locus includes a Bxb1 attB site or a modified attB* site. Other safe harbor loci may be used as provided herein.

The Bxb1 attachment site(s), in some embodiments, is/are located in or near the start codon (ATG) of an endogenous gene. For example, the normal transcriptional regulatory elements of an endogenous gene may be “intercepted” by including a Bxb1 attachment site near the start codon of the gene, then integrating the gene of interest (via Bxb1 integrase) such that transcription of the gene of interest is under the control of the transcriptional regulatory elements of the endogenous gene.

To produce a Bxb1 landing pad animal, a (at least one) single-stranded DNA (ssDNA) donor may be used. This ssDNA donor includes the Bxb1 attachment site(s) (e.g., a Bxb1 attP site or a Bxb1 attB site) flanked by homology arms. In some embodiments, a ssDNA includes two Bxb1 attachment sites (e.g., a Bxb1 attP site and a modified Bxb1 attP site, or a Bxb1 attB site and a modified Bxb1 attB site). One homology arm is located to the left (5′) of the Bxb1 attachment site(s) (the left homology arm) and another homology arm is located to the right (3′) of the Bxb1 attachment site(s) (the right homology arm). Homology arms are regions of the ssDNA that are homologous to regions of genomic DNA located in the genomic (e.g., safe harbor) locus. These homology arms enable homologous recombination between the ssDNA donor and the genomic locus, resulting in insertion of the Bxb1 attachment site(s) into the genomic locus, as discussed below (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded.

Examples of a left and right homology arm targeting the Rosa26 locus are provided as SEQ ID NO: 4 and SEQ ID NO: 5, respectively. In some embodiments, the ssDNA donor comprises a nucleotide sequence of SEQ ID NO: 3 (a Bxb1 attP attachment site flanked by left and right homology arms targeting the Rosa26 locus). In some embodiments, the ssDNA donor comprises a nucleotide sequence of SEQ ID NO: 9 (a modified Bxb1 attP* attachment site flanked by left and right homology arms targeting the Rosa26 locus).

In some embodiments, a mouse and/or mouse embryo (or other animal or animal embryo) of the present disclosure includes a single Bxb1 attachment site in a genomic locus of the mouse/mouse embryo. For example, the Bxb1 attachment site may be selected from attP attachment sites, modified attP* attachment sites, attB attachment sites, and modified attB* attachment sites.

In other embodiments, a mouse and/or mouse embryo (or other animal or animal embryo) of the present disclosure includes two (at least two) Bxb1 attachment sites in a genomic locus of the mouse/mouse embryo, which may be referred to herein as a first Bxb1 attachment site and a second Bxb1 attachment site. The first and second Bxb1 attachment sites, in some embodiments, are selected from attP attachment sites, modified attP* attachment sites, attB attachment sites, and modified attB* attachment sites. The first and second Bxb1 attachment sites may be adjacent to each other (with no intervening nucleotide sequence) or they may be separated from each other by a certain number of nucleotides. The number of nucleotides separating the two Bxb1 attachment sites may vary, provided, in some embodiments, that each Bxb1 attachment site is within the same safe harbor locus (e.g., within the Rosa26 locus). Thus, in some embodiments, any two (e.g., a first and second) Bxb1 attachments sites are separated from each other by at least 1, at least 2, at least 5, at least 10, at least 25, at least 50, at least 100, at least 150, at least 200, at least 250, at least 300, at least 350, at least 400, at least 450, at least 500, at least 1000, at least 1500, or at least 2000 nucleotide base pairs (bp). In some embodiments, any two (e.g., a first and second) Bxb1 attachments sites are separated from each other by 1 to 500 bp, 1 to 1000 bp, 1 to 1500 bp, 1 to 2000 bp, 1 to 2500 bp, or 1 to 3000 nucleotide base pairs (bp). For example, any two Bxb1 attachments sites may be separated from each other by 1 to 450 bp, 1 to 400 bp, 1 to 350 bp, 1 to 300 bp, 1 to 250 bp, 1 to 200 bp, 1 to 150 bp, 1 to 100 bp, 1 to 50 bp, 5 to 450 bp, 5 to 400 bp, 5 to 350 bp, 5 to 300 bp, 5 to 250 bp, 5 to 200 bp, 5 to 150 bp, 5 to 100 bp, 5 to 50 bp, 10 to 450 bp, 10 to 400 bp, 10 to 350 bp, 10 to 300 bp, 10 to 250 bp, 10 to 200 bp, 10 to 150 bp, 10 to 100 bp, 10 to 50 bp, 50 to 450 bp, 50 to 400 bp, 50 to 350 bp, 50 to 300 bp, 50 to 250 bp, 50 to 200 bp, 50 to 150 bp, 50 to 100 bp, 100 to 450 bp, 100 to 400 bp, 100 to 350 bp, 100 to 300 bp, 100 to 250 bp, 100 to 200 bp, or 100 to 150 bp.

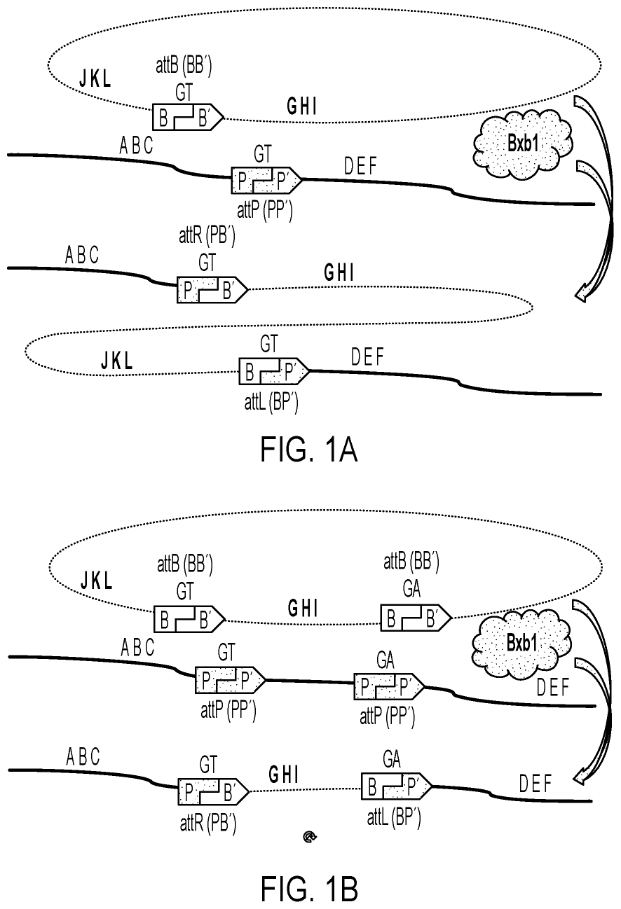

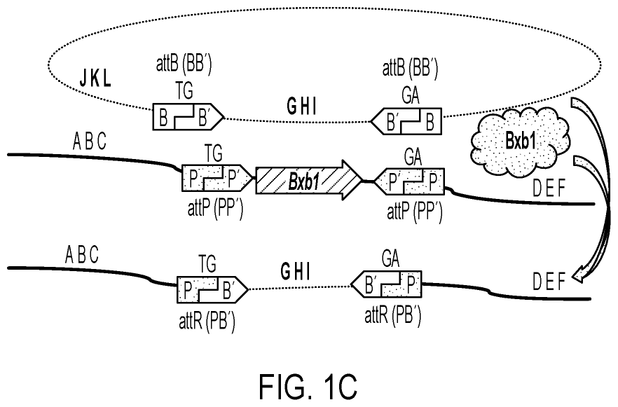

In some embodiments, an animal provided herein includes a polynucleotide, such as a genomic polynucleotide, that encodes a Bxb1 integrase. In such embodiments, the polynucleotide may be flanked by Bxb1 attachments sites such that the polynucleotide is removed following expression of the integrase and genomic integration of the gene of interest (see, e.g., FIG. 1C).

In some embodiments, insertion of a ssDNA donor comprising the Bxb1 attachment site(s) is facilitated by Clustered Regularly Interspaced Short Palindromic Repeats (CRISPR)/Cas9 gene editing. The CRISPR-Cas9 system is a naturally-occurring defense mechanism in prokaryotes that has been repurposed as a RNA-guided DNA-targeting platform used for gene editing. It relies on the DNA nuclease Cas9 (CRISPR associated protein 9) and a noncoding guide RNA (gRNA) to target the cleavage of DNA.

In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC).

A guide RNA (gRNA) directs the activities of an associated RNA-guided nuclease (e.g., Cas9) to a specific target sequence within a targeted genome. See, e.g., Jinek et al., Science, 337, 816-821 (2012) and Deltcheva et al., Nature, 471, 602-607 (2011). A gRNA comprises at least a spacer sequence that hybridizes to a target sequence (at a target site), and a CRISPR repeat sequence. In Type II systems (e.g., Streptococcus pyogenes systems), the gRNA comprises a tracrRNA (trans-activating RNA) sequence. In the Type II system, the CRISPR repeat sequence and tracrRNA sequence hybridize to each other to form a duplex. In the Type V system, a crRNA (CRISPR RNA) sequence forms a duplex. In both systems, the duplex binds a RNA-guided nuclease (e.g., Cas9) such that the gRNA and the RNA-guided nuclease form a complex. In some embodiments, the gRNA provides target specificity to the complex by virtue of its association with the RNA-guided nuclease. The gRNA thus directs the activity of the RNA-guided nuclease. Examples of gRNA spacer regions targeting the Rosa26 locus are provided as SEQ ID NO: 6 and SEQ ID NO: 10.

Other genome editing technologies may be used, such as transcription activator-like effector nucleases (TALENs) and/or zinc finger nucleases (ZFNs). See, e.g., Joung J K et al. Nat Rev Mol Cell Biol. 2013; 14(1):49-55; Carroll D Genetics. 2011; 188(4): 773-782; and Gaj T et al. Trends Biotechnol. 2013; 31(7):397-405.

Transgenic mice are most commonly produced by microinjection (pronuclear injection) of DNA into the pronuclei of fertilized single-cell (1-cell) mouse embryos. If DNA integration takes place prior to the first nuclear division, then some or all cells will carry the transgene. After injection, the eggs are surgically transferred to the oviducts of time-mated pseudopregnant foster mothers, generated by mating females with vasectomized males. The offspring resulting from injected eggs that carry the transgene are called founders.

While microinjection is exemplified, other transfection systems may be used to produce the BXb1 landing pad animals of the present disclosure, such as, for example, electroporation (see, e.g., Wang W et al. J Genet Genomics 2016; 43(5):319-27), embryonic stem cell-mediated gene transfer and retrovirus-mediated gene transfer (see, e.g., Kumar T R et al. Methods Mol Biol. 2009; 590:335-362), and viral-based gene transfer.

In some embodiments, gene transfer takes place at the single-cell stage of an embryo, which may be referred to as the zygote. In other embodiments, gene transfer takes place at a later multicellular stage (two or more cells, also called blastomeres) of an embryo. In some embodiments, pronuclear microinjection occurs at the zygote stage, and is followed by nuclear injection at the two-cell stage (twice).

In some embodiments, animal (e.g., mammal, such as rodent, for example, mouse) strains that express Cre recombinase-dependent Cas9 expression may be used. These mouse strains allow for in vivo CRISPR gene editing when a viral vector co-expressing Cre and the gRNA is injected. The virally-expressed Cre turns on Cas9 expression, which in turn edits the targeted gene or genes. Additionally, in vivo gene editing in mice can be accomplished by local or systemic injection of Cas9 and gRNA expressing lenti- or adeno-associated viruses.

Any mouse may be used to generate a Bxb1 landing pad strain. Non-limiting examples of mouse strains include C57BL/6J mice (664), C57BL/6NJ (5304), FVB/NJ (1800), B6D2 (C57BL/6×DBA/2J) mice, and NGS™ (NOD scid gamma) mice (5557) or variants thereof. Additional examples include A/J (000646), 129S1/SvImJ (002448), NOD/ShiLtJ (001976), NZO/HiLtJ (002105), CAST/EiJ (000928), PWK/PhJ (003715), WSB/EiJ (001145), DBA2 (000671), and Collaborative Cross (CC) strains.

In some embodiments, a method of producing a Bxb1 landing pad animal (e.g., mammal, such as rodent, for example, mouse) may comprise isolating a fertilized single-cell embryo, and microinjecting the pronucleus or cytoplasm of the embryo with Cas9 (e.g., Cas9 protein, or DNA or mRNA encoding Cas9 protein), a gRNA (or DNA encoding the gRNA), and a ssDNA targeting a genomic locus, such as a safe harbor locus (e.g., the Rosa26 locus or other open chromatin locus). Microinjected embryos may then be transferred to pseudopregnant female mice and carried to term. Pseudopregnancy describes a false pregnancy whereby all the signs and symptoms of pregnancy are exhibited, with the exception of the presence of a zygote. Mice become pseudopregnant following an estrus in which the female is bred by an infertile male, resulting in sterile mating. At ˜2-3 weeks of age, tail biopsies may be collected from the offspring and correct integration into the safe harbor locus may be verified by polymerase chain reaction (PCR), sequencing, Southern blotting and/or long-range sequencing systems, e.g., PacBio. Founder mice that carry the desired integration are then bred to generate a Bxb1 landing pad mouse strain.

### Targeted Transgene Integration

The Bxb1 landing pad animal (e.g., mammal, such as rodent, for example, mouse) may be used, in some embodiments, to introduce a gene of interest at the Bxb1 attachment site of the animal genome. In some embodiments, the gene of interest is present on a vector. A vector is simply a DNA molecule that is used as a vehicle to carry exogenous genetic material (e.g., donor transgene) into a host cell (e.g., mouse embryo). In some embodiments, a gene of interest is present on a circular donor polynucleotide, such as a plasmid. In some embodiments, for example, when using an animal that includes only one Bxb1 attachment site in its genome, the circular donor polynucleotide is a DNA minicircle. DNA minicircles are small (— 4 kb) circular vector backbone with donor DNA to be circularized of >100 bp to 50 kb. In some embodiments, a DNA minicircle is a plasmid derivative that has been freed from all prokaryotic vector parts (e.g., no longer contains a bacterial plasmid backbone comprising antibiotic resistance markers and/or bacterial origins of replication).

Methods of producing DNA minicircles are well-known in the art. For example, a parental plasmid that comprises a bacterial backbone and the eukaryotic inserts, including the transgene to be expressed, may be produced in a specialized E. coli strain that expresses a site-specific recombinase protein. Recombination sites flank the eukaryotic inserts in the parental plasmid, so that when the activity of the recombinase protein (non-Bxb1) is induced by methods such as, but not limited to, arabinose induction, glucose induction, etc., the bacterial backbone is excised from the eukaryotic insert, resulting in a eukaryotic DNA minicircle and a bacterial plasmid.

The sequence (e.g., gene) of interest, in some embodiments, has a length of 200 base pairs (bp) to 100 kilobases (kb). The gene of interest, in some embodiments, has a length of at least 10 kb. For example, the gene of interest may have a length of at least 15 kb, at least 20 kb, at least 25 kb, at least 30 kb, or at least 35 kb. In some embodiments, the gene of interest has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

The donor polynucleotide(s), in some embodiments, has a length of 200 bp to 500 kb, 200 bp to 250 kb, or 200 bp to 100 kb. The donor polynucleotide, in some embodiments, has a length of at least 10 kb. For example, the donor polynucleotide may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, the donor polynucleotide has a length of 10 to 500 kb, 20 to 400 kb, 10 to 300 kb, 10 to 200 kb, or 10 to 100 kb. In some embodiments, the donor polynucleotide has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A donor polynucleotide may be circular or linear.

In some embodiments, a donor polynucleotide(s) comprising a gene of interest and the corresponding (cognate) Bxb1 attachment site(s) is introduced into (e.g., via microinjection) an embryo, such as a single-cell embryo (zygote). Later-stage embryos or animals may also be used. Pronucleus microinjection and other gene transfer methods for use as provided herein are discussed above.

The donor polynucleotide(s), in some embodiments, is introduced into an embryo or animal with a Bxb1 integrase protein, a polynucleotide encoding a Bxb1 integrase protein, or a Bxb1 integrase protein and a polynucleotide encoding a Bxb1 integrase protein. The polynucleotide may be DNA or RNA (e.g., mRNA).

Following introduction of the donor polynucleotide and the Bxb1 integrase into an embryo, the embryo may be implanted into a pseudopregnant female to produce genetically-modified progeny animals comprising the gene of interest, similar to the breeding process described above.

In some embodiments, at least 10% of the genetically-modified progeny animals comprises the gene of interest integrated into the genomic locus. In some embodiments, at least 11%, at least 12%, at least 13%, at least 14%, or at least 15% of the genetically-modified progeny animals comprises the gene of interest integrated into the genomic locus. In some embodiments, at least 15%, at least 20%, at least 30%, at least 35%, at least 40%, at least 45%, or at least 50% of the genetically-modified progeny animals comprises the gene of interest integrated into the genomic locus. For example, 10% to 50%, 10% to 40%, 10% to 30%, or 10% to 20% of the genetically-modified progeny animals comprises the gene of interest integrated into the genomic locus. In some embodiments, greater than 50% (e.g., 55%, 60%, 65%, or 70%) of the genetically-modified progeny animals comprises the gene of interest integrated into the genomic locus.

In some embodiments, the gene of interest (or donor polynucleotide, which includes the gene of interest) has a length of at least 10 kb and at least 10% of the genetically-modified progeny animals comprises the gene of interest integrated correctly into the genomic locus. For example, the gene of interest may have a length of at least 10 kb, at least 15 kb, at least 20 kb, at least 25 kb, at least 30 kb, at least 35 kb, at least 40 kb, at least 45 kb, or at least 50 kb, and at least 10%, at least 15%, at least 20%, at least 25%, at least 30%, at least 35%, at least 40%, at least 45%, or at least 50% of the genetically-modified progeny animals comprises the gene of interest integrated into the genomic locus.

In some embodiments, the gene of interest has a length of at least 10 kb and at least 15% of the genetically-modified progeny animals comprises the gene of interest integrated into the genomic locus. In some embodiments, the gene of interest has a length of at least 10 kb and at least 20% of the genetically-modified progeny animals comprises the gene of interest integrated into the genomic locus. In some embodiments, the gene of interest has a length of at least 10 kb and at least 25% of the genetically-modified progeny animals comprises the gene of interest integrated into the genomic locus. In some embodiments, the gene of interest has a length of at least 10 kb and at least 30% of the genetically-modified progeny animals comprises the gene of interest integrated into the genomic locus.

In some embodiments, the gene of interest has a length of at least 15 kb and at least 15% of the genetically-modified progeny animals comprises the gene of interest integrated into the genomic locus. In some embodiments, the gene of interest has a length of at least 15 kb and at least 20% of the genetically-modified progeny animals comprises the gene of interest integrated into the genomic locus. In some embodiments, the gene of interest has a length of at least 15 kb and at least 25% of the genetically-modified progeny animals comprises the gene of interest integrated into the genomic locus. In some embodiments, the gene of interest has a length of at least 15 kb and at least 30% of the genetically-modified progeny animals comprises the gene of interest integrated into the genomic locus.

In some embodiments, the gene of interest has a length of at least 20 kb and at least 15% of the genetically-modified progeny animals comprises the gene of interest integrated into the genomic locus. In some embodiments, the gene of interest has a length of at least 20 kb and at least 20% of the genetically-modified progeny animals comprises the gene of interest integrated into the genomic locus. In some embodiments, the gene of interest has a length of at least 20 kb and at least 25% of the genetically-modified progeny animals comprises the gene of interest integrated into the genomic locus. In some embodiments, the gene of interest has a length of at least 20 kb and at least 30% of the genetically-modified progeny animals comprises the gene of interest integrated into the genomic locus.

In some embodiments, the gene of interest has a length of at least 25 kb and at least 15% of the genetically-modified progeny animals comprises the gene of interest integrated into the genomic locus. In some embodiments, the gene of interest has a length of at least 25 kb and at least 20% of the genetically-modified progeny animals comprises the gene of interest integrated into the genomic locus. In some embodiments, the gene of interest has a length of at least 25 kb and at least 25% of the genetically-modified progeny animals comprises the gene of interest integrated into the genomic locus. In some embodiments, the gene of interest has a length of at least 25 kb and at least 30% of the genetically-modified progeny animals comprises the gene of interest integrated into the genomic locus.

In some embodiments, the gene of interest has a length of at least 30 kb and at least 15% of the genetically-modified progeny animals comprises the gene of interest integrated into the genomic locus. In some embodiments, the gene of interest has a length of at least 30 kb and at least 20% of the genetically-modified progeny animals comprises the gene of interest integrated into the genomic locus. In some embodiments, the gene of interest has a length of at least 30 kb and at least 25% of the genetically-modified progeny animals comprises the gene of interest integrated into the genomic locus. In some embodiments, the gene of interest has a length of at least 30 kb and at least 30% of the genetically-modified progeny animals comprises the gene of interest integrated into the genomic locus.

In some embodiments, the gene of interest has a length of at least 35 kb and at least 15% of the genetically-modified progeny animals comprises the gene of interest integrated into the genomic locus. In some embodiments, the gene of interest has a length of at least 35 kb and at least 20% of the genetically-modified progeny animals comprises the gene of interest integrated into the genomic locus. In some embodiments, the gene of interest has a length of at least 35 kb and at least 25% of the genetically-modified progeny animals comprises the gene of interest integrated into the genomic locus. In some embodiments, the gene of interest has a length of at least 35 kb and at least 30% of the genetically-modified progeny animals comprises the gene of interest integrated into the genomic locus.

### Additional Embodiments

Additional embodiments are encompassed by the numbered paragraphs below.

1. A mammal comprising within its genome a first Bxb1 attachment site and a second Bxb1 attachment site.
2. The mammal of paragraph 1 further comprising a polynucleotide encoding a Bxb1 integrase, optionally wherein the polynucleotide is flanked by the first and second Bxb1 attachment sites.
3. The mammal of paragraph 1 or 2, wherein

the first Bxb1 attachment site is selected from an attP site, a modified attP* site, an attB site, and a modified attB* site;

the second Bxb1 attachment site is selected from an attP site, a modified attP* site, an attB site, and a modified attB* site; and

optionally the first and second Bxb1 attachment sites are heterologous relative to each other, and optionally the genome does not include attR and/or attL sites, for example, separated from each other by 50 to 500 nucleotide bases.

4. The mammal of paragraph 3, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.
5. The mammal of any one of paragraphs 1-4, wherein the first and second Bxb1 attachment sites are separated from each other by 50 to 500 nucleotide bases.
6. The mammal of any one of the preceding paragraphs, wherein the first and second Bxb1 attachment sites are within a safe harbor locus, optionally a Rosa26 locus.
7. The mammal of any one of the preceding paragraphs, wherein the mammal is a rodent, optionally a mouse.
8. A mammalian embryo comprising within its genome a first Bxb1 attachment site and a second Bxb1 attachment site.
9. The mammalian embryo of paragraph 8 further comprising (a) a Bxb1 integrase or (b) a polynucleotide encoding a Bxb1 integrase, optionally wherein the polynucleotide is flanked by the first and second Bxb1 attachment sites.
10. The mammalian embryo of paragraph 8 or 9, wherein

the first Bxb1 attachment site is selected from an attP site, a modified attP* site, an attB site, and a modified attB* site;

the second Bxb1 attachment site is selected from an attP site, a modified attP* site, an attB site, and a modified attB* site; and

optionally the first and second Bxb1 attachment sites are heterologous relative to each other and optionally the genome does not include attR and/or attL sites, for example, separated from each other by 50 to 500 nucleotide bases.

11. The mammalian embryo of paragraph 10, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.
12. The mammalian embryo of any one of paragraphs 8-11, wherein the first and second Bxb1 attachment sites are separated from each other by 50 to 500 nucleotide base pairs.
13. The mammalian embryo of any one of the preceding paragraphs, wherein the first and second Bxb1 attachment sites are within a safe harbor locus, optionally a Rosa26 locus.
14. The mammalian embryo of any one of the preceding paragraphs, wherein the mammalian embryo is a single-cell embryo or a multi-cell embryo.
15. The mammalian embryo of any one of the preceding paragraphs, wherein the mammalian embryo is a rodent embryo, optionally a mouse embryo.
16. A method comprising

introducing into the mammalian embryo of any one of the preceding paragraphs (a) a donor polynucleotide comprising a sequence of interest flanked by a first cognate Bxb1 attachment site and a second cognate Bxb1 attachment site, and (b) a Bxb1 integrase or a polynucleotide encoding a Bxb1 integrase.

17. A method comprising

introducing into the mammalian embryo of any one of the preceding paragraphs a donor polynucleotide comprising a sequence of interest flanked by a first cognate Bxb1 attachment site and a second cognate Bxb1 attachment site.

18. The method of paragraph 16 or 17 further comprising implanting the mammalian embryo into a pseudopregnant female mammal.
19. The method of paragraph 18 further comprising collecting from the female mammal a progeny mammal.
20. The method of paragraph 19 further comprising screening the progeny mammal for presence or absence of the sequence of interest integrated into the genome of the progeny mammal.
21. The method of any one of the preceding paragraphs, wherein the donor polynucleotide, the Bxb1 integrase, and/or the polynucleotide encoding a Bxb1 integrase is introduced into the mammalian embryo via microinjection.
22. The method of any one of the preceding paragraphs, wherein the sequence of interest comprises a gene of interest.
23. The method of any one of the preceding paragraphs, wherein the sequence of interest has a size of at least 10 kb, at least 15 kb, at least 20 kb, at least 25 kb, or at least 30 kb.
24. A method for producing a Bxb1 landing pad mammal, the method comprising:

(a) introducing into a mammalian embryo (i) a Cas9 nuclease or a polynucleotide encoding a Cas9 nuclease, (ii) a first guide RNA (gRNA) or a polynucleotide encoding a gRNA that targets a first genomic site in the mammalian embryo, (iii) a first single-stranded DNA (ssDNA) donor comprising a first Bxb1 attachment site flanked by a left homology arm and a right homology arm; optionally (iv) a second guide RNA (gRNA) or a polynucleotide encoding a gRNA that targets a second genomic in the mammalian embryo, and (v) a second ssDNA comprising a second Bxb1 attachment site flanked by a left homology arm and a right homology arm; and

(b) implanting the mammalian embryo cell into a pseudopregnant female mammal, wherein the pseudopregnant female mammal is capable of giving birth to a progeny mammal.

25. The method of paragraph 24, wherein the first ssDNA further comprises a second Bxb1 attachment site upstream or downstream from the first Bxb1 attachment site, wherein both the first and second Bxb1 attachment sites are flanked by the left homology arm and the right homology arm.
26. The method of paragraph 24 or 25, wherein the mammalian embryo comprises a Bxb1 integrase or polynucleotide encoding a Bxb1 integrase, or step (a) further comprises introducing into the mammalian embryo a Bxb1 integrase or a polynucleotide encoding a Bxb1 integrase.
27. The method of any one of paragraphs 24-26 further comprising collecting the progeny mammal.
28. The method of any one of paragraphs 24-27, wherein the mammalian embryo is a rodent embryo, optionally a mouse embryo.
29. A mammal comprising the mammalian embryo of paragraph 8-14.
30. The mammal of paragraph 29, wherein the mammal is a rodent, optionally a mouse.
31. A mammal comprising within its genome a Bxb1 attachment site.
32. The mammal of paragraph 31 further comprising a Bxb1 integrase or a polynucleotide encoding a Bxb1 integrase.
33. The mammal of paragraph 31 or 32, wherein the Bxb1 attachment site is an attP site, a modified attP* site, an attB site, or a modified attB* site.
34. The mammal of paragraph 33, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.
35. The mammal of any one of the preceding paragraphs, wherein the Bxb1 attachment site is within a safe harbor locus, optionally a Rosa26 locus
36. The mammal of any one of the preceding paragraphs, wherein the mammal is a rodent, optionally a mouse.
37. A mammalian embryo comprising within its genome a Bxb1 attachment site.
38. The mammalian embryo of paragraph 37 further comprising a Bxb1 integrase or a polynucleotide encoding a Bxb1 integrase.
39. The mammalian embryo of paragraph 37 or 38, wherein the Bxb1 attachment site is an attP site, a modified attP* site, an attB site, or a modified attB* site.
40. The mammalian embryo of paragraph 39, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.
41. The mammalian embryo of any one of the preceding paragraphs, wherein the Bxb1 attachment site is within a safe harbor locus, optionally a Rosa26 locus.
42. The mammalian embryo of any one of the preceding paragraphs, wherein the mammalian embryo is a single-cell embryo or a multi-cell embryo.
43. The mammalian embryo of any one of the preceding paragraphs, wherein the mammalian embryo is a rodent embryo, optionally a mouse embryo.
44. A method comprising

introducing into the mammalian embryo of any one of paragraphs 37-43 (a) a donor polynucleotide comprising a sequence of interest and a cognate Bxb1 attachment site and (b) a Bxb1 integrase or a polynucleotide encoding a Bxb1 integrase.

45. A method comprising

introducing into the mammalian embryo of any one of paragraphs 37-43 a donor polynucleotide comprising a sequence of interest and a cognate Bxb1 attachment site.

46. The method of paragraph 44 or 45 further comprising implanting the mammalian embryo into a pseudopregnant female mammal.
47. The method of paragraph 46 further comprising collecting from the female mammal a progeny mammal.
48. The method of paragraph 47 further comprising screening the progeny mammal for presence or absence of the sequence of interest integrated into the genome of the progeny mammal.
49. The method of any one of the preceding paragraphs, wherein the donor polynucleotide, the Bxb1 integrase, and/or the polynucleotide encoding a Bxb1 integrase is introduced into the mammalian embryo via microinjection.
50. The method of any one of the preceding paragraphs, wherein the donor polynucleotide is a minicircle.
51. The method of any one of the preceding paragraphs, wherein the sequence of interest comprises a gene of interest.
52. The method of any one of the preceding paragraphs, wherein the sequence of interest has a size of at least 3 kb, at least 4 kb, at least 5 kb, at least 6 kb, at least 7 kb, at least 8 kb, at least 9 kb, or at least 10 kb.
53. A method for producing a Bxb1 landing pad mammal, the method comprising:

(a) introducing into a mammalian embryo
(i) a Cas9 nuclease or a polynucleotide encoding a Cas9 nuclease,
(ii) a guide RNA (gRNA) or a polynucleotide encoding a gRNA that targets a genomic locus in the mammalian embryo, and
(iii) a single-stranded DNA (ssDNA) donor comprising a Bxb1 attachment site flanked by a left homology arm and a right homology arm; and

(b) implanting the mammalian embryo cell into a pseudopregnant female mammal, wherein the pseudopregnant female mammal is capable of giving birth to a progeny mammal.

54. The method of paragraph 53, wherein the mammalian embryo comprises a polynucleotide encoding a Bxb1 integrase, or step (a) further comprises introducing into the mammalian embryo a polynucleotide encoding a Bxb1 integrase.
55. The method of paragraph 54 further comprising collecting the progeny mammal.
56. The method of paragraph 55, wherein the mammalian embryo is a rodent embryo, optionally a mouse embryo.
57. A mammal comprising the mammalian embryo of paragraph 37-43.
58. The mammal of paragraph 57, wherein the mammal is a rodent, optionally a mouse.

### EXAMPLES

### Example 1. Bxb1 Mouse—One (attP) Landing Pad

Using CRISPR/Cas9 and an oligonucleotide donor, we inserted a single attP site into the Rosa26 locus of the mouse genome (a C57BL/6J, NSG™, PWK/PhJ, DBA/2J, A/J, 129S1/SvImJ, or FVB/NJ mouse line).

Fertilized zygotes were isolated from C57BL/6J mice. The pronuclei of these zygotes were microinjected with (1) Cas9 as mRNA, or protein, or both mRNA and protein (concentrations ranged from 60-100 ng/μL for mRNA and 30-60 ng/μL for protein), (2) gRNA (concentrations ranged from 30-50 ng/μL), and (3) a ssDNA oligo of approximately 200 bp targeting the Rosa26 locus (SEQ ID NO: 3). This ssDNA oligo has 152 bases of homology (SEQ ID NOs: 4 and 5) flanking a 48-base pair Bxb1 attP site (SEQ ID NO: 2). Microinjected zygotes were transferred to pseudopregnant mice and carried to term. At ˜2-3 weeks of age, tail biopsies were collected from offspring and tested by PCR and sequencing for the correct integration of the attP site. Mice carrying the Bxb1 attP site were bred to create a Bxb1 attP mouse strain.

These mice were then used as recipients for integrase-mediated recombination with donors that contained a matching cognate attB site (FIGS. 2A-2B). In order to avoid the insertion of DNA from the vector backbone, plasmids were converted into minicircles (System Biosciences, LLC) prior to microinjection.

Fertilized zygotes were isolated from the Bxb1 attP C57BL/6J mouse strain. The pronuclei of these zygotes were microinjected with mRNA encoding Bxb1 integrase at 100 ng/μL and the donor DNA at 1-10 ng/μL. The donor DNA carries a Bxb1 attB site matching the host embryo Bxb1 attP site. The donor DNA was a bacterial vector free minicircle using technologies well known and established in the field. The microinjected zygotes were transferred to pseudopregnant mice and carried to term. At >2-3 weeks of age, tail biopsies were collected from offspring and tested for the correct integration of the DNA by PCR and sequencing.

Results with this version are outlined in Table 1.

TABLE 1
Summary of Results

| DNA | INSERT | | | | EFFICIENCY |
|---|---|---|---|---|---|
| INSERT | SIZE | STRAIN | | EFFICIENCY | (SUCCESS/ |
| ID | (KB) | BACKGROUND | PASS/FAIL | (TG/LIVEBORN) | ATTEMPT) |
| MCP5072 | 3.3 | NSG | FAIL | 0/29 (0%) | 0/1 (0%) |
| MCP5098 | 3.9 | B6 | FAIL | 0/71 (0%) | 0/2 (0%) |
| MCP5172 | 7.7 | 129S1 | FAIL | 0/23 (0%) | 0/2 (0%) |
| MCP5172 | 7.7 | NSG | FAIL | 0/20 (0%) | 0/2 (0%) |
| MCP5114 | 3.2 | B6 | PASS | 2/67 (3%) | 1/1 (100%) |
| MCP5073 | 3.3 | NSG | PASS | 10/157 (6%) | 5/10 (50%) |
| MCP5113A | 3.8 | B6 | PASS | 2/48 (4%) | 1/1 (100%) |
| MCP5134 | 4.0 | B6 | PASS | 2/50 (4%) | 1/2 (50%) |
| MCP5092 | 7.5 | B6 | PASS | 3/19 (16%) | 1/1 (100%) |
| MCP5093 | 7.5 | B6 | PASS | 2/38 (5%) | 1/1 (100%) |
| MCP5172 | 7.7 | B6 | PASS | 1/43 (2%) | 1/3 (33%) |
| MCP5172 | 7.7 | FVB | PASS | 2/113 (2%) | 1/3 (33%) |
| MCP5117 | 9.9 | B6 | PASS | 1/99(1%) | 1/2 (50%) |
| | | | Projects | Embryos | MIJ's |
| MIN = | 3.2 | | 9/13 (69%) | 31/777 (4%) | 13/31 (42%) |
| MAX = | 9.9 | | | | |

TG = transgene positive

### Example 2. Bxb1 Mouse—Two (attP) Landing Pads

We generated our Version 2 recipient mice through sequential modification of the Version 1 lines. Again, we used CRISPR/Cas9 and an oligonucleotide donor to insert a second attP* (modified) site ˜240 bp away from the original attP site. In the modified site, the dinucleotide pairing was changed from GT to GA. The addition of the second site allows for exclusion of the vector backbone without first having to convert the donor construct into minicircles (FIG. 1B). Note that while in this example the two attachment sites were inserted sequentially, they could be inserted simultaneously using a single donor polynucleotide, for example. The Version 2 mouse also makes it possible to insert even larger tracts of DNA, as the requisite attB sites can be placed into any vector (including BACs) by simply flanking the desired region to be integrated.

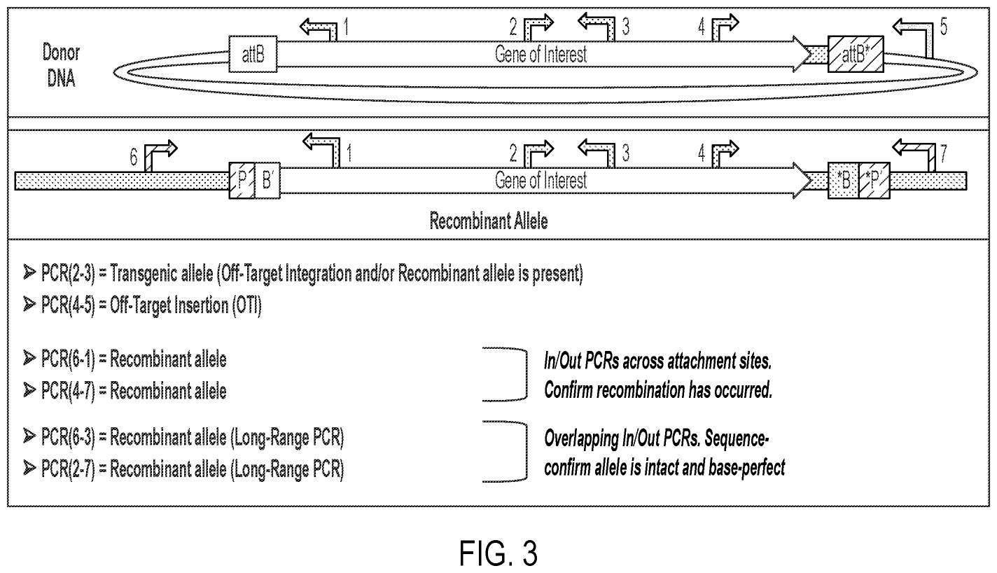

Screening and verification can be one of the greatest challenges when creating mice with large knock-ins. However, knowing the precise location and orientation of the transgene allows for rapid and straightforward identification by PCR. A general strategy for screening is outlined in FIG. 3.

We tested the Version 2 mouse using a recombineered BAC (total size 33,939 bp) to insert a 30,570 bp tract of human genomic DNA. The results are summarized in Table 2.

TABLE 2
Summary of Results

| Background Strain | Size of Insert | Recombination Rate |
|---|---|---|
| C57BL/6J | 30.6 kb | 11% (4/35) |

We have tested the Version 2 mouse inserting nucleic acids having various lengths, ranging from 1.5 kb to 30.6 kb. The results are summarized in Table 3.

TABLE 3
Summary of Results

| | INSERT | | | | EFFICIENCY |
|---|---|---|---|---|---|
| DNA | SIZE | STRAIN | | EFFICIENCY | (SUCCESS/ |
| INSERT ID | (KB) | BACKGROUND | PASS/FAIL | (TG/LIVEBORN) | ATTEMPT) |
| BAC(GET4781) | 87.1 | NSG | FAIL | 0/31 (0%) | 0/2 (0%) |
| P5178 | 1.5 | B6 | PASS | 9/23 (39%) | 1/1 (100%) |
| P5161 | 3.4 | B6 | PASS | 2/111 (2%) | 1/2 (50%) |
| P5162 | 3.4 | B6 | PASS | 6/103 (6%) | 1/2 (50%) |
| P5169 | 3.7 | B6 | PASS | 9/48 (19%) | 1/1 (100%) |
| P5167 | 4.6 | B6 | PASS | 13/44 (30%) | 1/1 (100%) |
| P5166 | 5.7 | B6 | PASS | 6/50 (12%) | 1/1 (100%) |
| P5168 | 5.8 | B6 | PASS | 7/39 (18%) | 1/1 (100%) |
| P5183 | 6.8 | B6 | PASS | 11/54 (20%) | 1/1 (100%) |
| P5183 | 6.8 | NSG | PASS | 19/60 (32%) | 1/1 (100%) |
| P5188 | 7.1 | B6 | PASS | 3/41 (7%) | 1/2 (50%) |
| P5151 | 8.8 | B6 | PASS | 1/14 (7%) | 1/1 (100%) |
| P5175 | 9.1 | B6 | PASS | 9/20 (45%) | 1/1 (100%) |
| P5148 | 9.4 | B6 | PASS | 8/64 (13%) | 1/2 (50%) |
| P5187 | 10.3 | B6 | PASS | 2/11 (18%) | 1/1 (100%) |
| P5136 | 30.6 | B6 | PASS | 4/35 (11%) | 1/1 (100%) |
| | | | Projects | Embryos | MIJ's |
| MIN = | 1.5 | | 15/16 (94%) | 108/748 (14%) | 15/21 (71%) |
| MAX = | 30.6 | | | | |

TG = transgene positive

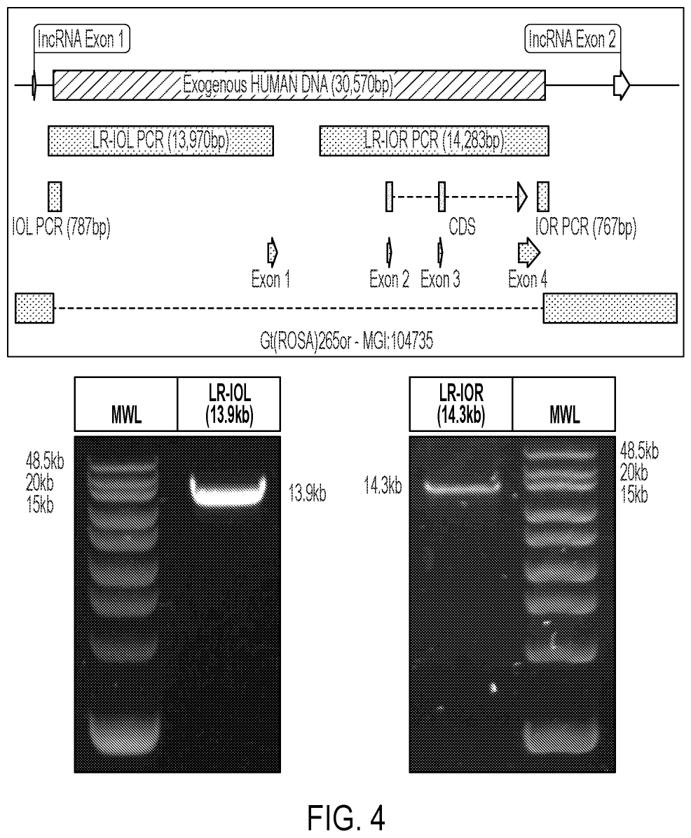

The four founder candidates were back-crossed to wild-type mice and the N1 offspring were assessed for germ-line transmission of both the desired recombinant allele (REC) as well as any off-target insertion (OTI) events. Offspring from two of the four founder lines also harbored an undesired random transgenic allele. The OTI and REC alleles segregated, indicating the insertion events are not linked. Long-Range PCR was performed to confirm the integrated allele is intact (FIG. 4).

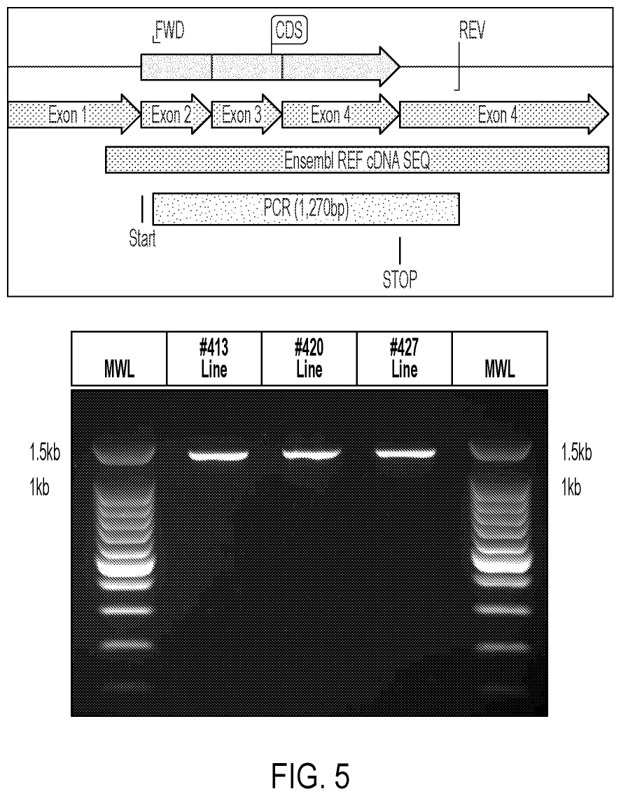

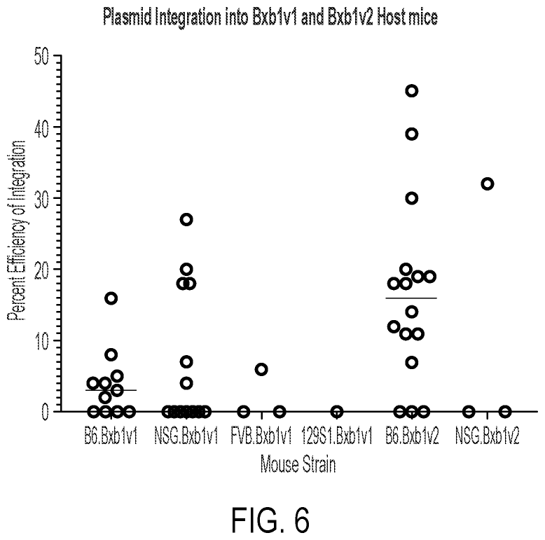

As the transgene is known to have a high expression in human liver, RNA was isolated from the livers of mice from three of the four colonies (the colony from the fourth founder had yet to reach sufficient size so it was not included in these tests). cDNA was prepared from total RNA and a 1,270 bp PCR product was generated from each line (FIG. 5). Sanger sequencing of this product was then used to confirm that the transcript is in fact, the humanized allele.

SEQUENCES

Bxb1 attP site
GGTTTGTCTGGTCAACCACCGCG [symbol] CTCAGTGGTGTACGGTACAAACC
(SEQ ID NO: 1)
Bxb1 attP* site
GGTTTGTCTGGTCAACCACCGCG [symbol] CTCAGTGGTGTACGGTACAAACC
(SEQ ID NO: 7)
Bxb1 attB site
GGCTTGTCGACGACGGCG [symbol] CTCCGTCGTCAGGATCAT (SEQ ID
NO: 2)
Bxb1 attB* site
GGCTTGTCGACGACGGCG [symbol] CTCCGTCGTCAGGATCAT (SEQ ID
NO: 8).
ssDNA targeting the Rosa26 locus - Bxb1 attP site
GAGGACCGCCCTGGGCCTGGGAGAATCCCTTCCCCCTCTTCCCTCGTGAT
CTGCAACTCCAGTCTTTCTAGAAGATGGTTTGTCTGGTCAACCACCGCG [symbol]
GTCTCAGTGGTGTACGGTACAAACCGGGCGGGAGTCTTCTGGGCAGGC
TTAAAGGCTAACCTGGTGTGTGGGCGTTGTCCTGCAGGGGAATTGAACAG
GTG (SEQ ID NO: 3)
ssDNA targeting the Rosa26 locus - Bxb1 attP* site
GCAAAACTACAGGTTATTATTGCTTGTGATCCGCCTCGGAGTATTTTCCA
TCGGGTTTGTCTGGTCAACCACCGCG [symbol] CTCAGTGGTGTACGGTACAAAC
CAGGTAGATTAAAGACATGCTCACCCGAGTT (SEQ ID NO: 9)
C57BL6/J Rosa26 locus, 5′ left homology arm for
insertion of Bxb1 attP site
GAGGACCGCCCTGGGCCTGGGAGAATCCCTTCCCCCTCTTCCCTCGTGAT
CTGCAACTCCAGTCTTTCTAGAAGAT (SEQ ID NO: 4)
C57BL6/J Rosa26 locus, 3′ right homology arm for
insertion of Bxb1 attP site
GGGCGGGAGTCTTCTGGGCAGGCTTAAAGGCTAACCTGGTGTGTGGGCGT
TGTCCTGCAGGGGAATTGAACAGGTG (SEQ ID NO: 5)
gRNA targeting Rosa26 locus in C57BL/6J mouse
zygotes
GUCUUUCUAGAAGAUGGG (SEQ ID NO: 6)
gRNA targeting Rosa26 locus in C57BL/6J mouse
zygotes
GUCUUUAAUCUACCUCGA (SEQ ID NO: 10)

### REFERENCES

1. Ghosh, P. et. al. Synapsis in Phage Bxb1 Integration: Selection Mechanism for the Correct Pair of Recombination Sites. J. Mol. Biol. 349, 331-348 (2005).
2. Tasic, B. et. al. Site-specific integrase-mediated transgenesis in mice via pronuclear injection. PNAS 108, 7902-7907 (2011).
3. Fogg, P. C. M. et. al. New Applications for Phage Integrases. J. Mol. Biol. 426, 2703-2716 (2014).
4. Rossant, J. et. al. Engineering the embryo. PNAS 108, 7659-7660 (2011).
5. Xu, Z. et. al. Accuracy and efficiency define Bxb1 integrase as the best of fifteen candidate serine recombinases for the integration of DNA into the human genome. BMC Biotechnology 13, (2013).
6. Wu, J. et. al. Custom-designed zinc finger nucleases: What is next? Cell Mol. Life Sci. 64, 2933-2944 (2007).
7. Munye, M. M. et. al. Minicircle DNA Provides Enhanced and Prolonged Transgene Expression Following Airway Gene Transfer. Scientific Reports 6, (2016).
8. Russell, J. P. et. al. Phage Bxb1 integrase mediates highly efficient site-specific recombination in mammalian cells. BioTechniques 40, (2006).
9. Geisinger, J. M. et. al. Using phage integrases in a site-specific dual integrase cassette exchange strategy. Methods in Molecular Biology 1239, (2015).
10. Smitch, M. C. M., et. al. Site-specific recombination by phiC31 integrase and other large serine recombinases. Biochemical Society Transactions 38, (2010).
11. Mediavilla, J. et. al. Genome organization and characterization of mycobacteriophage Bxb1. Molecular Microbiology 38, (2000).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A mammal comprising within its genome a first Bxb1 attachment site and a second Bxb1 attachment site.

2. The mammal of claim 1 further comprising a polynucleotide encoding a Bxb1 integrase, optionally wherein the polynucleotide is flanked by the first and second Bxb1 attachment sites.

3. The mammal of claim 1, wherein
the first Bxb1 attachment site is selected from an attP site, a modified attP* site, an attB site, and a modified attB* site;
the second Bxb1 attachment site is selected from an attP site, a modified attP* site, an attB site, and a modified attB* site; and
optionally the first and second Bxb1 attachment sites are heterologous relative to each other.

4. The mammal of claim 3, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.

5. The mammal of claim 1, wherein the first and second Bxb1 attachment sites are separated from each other by 50 to 500 nucleotide bases.

6. The mammal of claim 1, wherein the first and second Bxb1 attachment sites are within a safe harbor locus, optionally a Rosa26 locus.

7. The mammal of claim 1, wherein the mammal is a rodent, optionally a mouse.

8. A mammalian embryo comprising within its genome a first Bxb1 attachment site and a second Bxb1 attachment site.

9. The mammalian embryo of claim 8 further comprising a polynucleotide encoding a Bxb1 integrase, optionally wherein the polynucleotide is flanked by the first and second Bxb1 attachment sites.

10. The mammalian embryo of claim 8, wherein
the first Bxb1 attachment site is selected from an attP site, a modified attP* site, an attB site, and a modified attB* site;
the second Bxb1 attachment site is selected from an attP site, a modified attP* site, an attB site, and a modified attB* site; and
optionally the first and second Bxb1 attachment sites are heterologous relative to each other.

11. The mammalian embryo of claim 10, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.

12. The mammalian embryo of claim 8, wherein the first and second Bxb1 attachment sites are separated from each other by 50 to 500 nucleotide base pairs.

13. The mammalian embryo of claim 8, wherein the first and second Bxb1 attachment sites are within a safe harbor locus, optionally a Rosa26 locus.

14. The mammalian embryo of claim 8, wherein the mammalian embryo is a single-cell embryo or a multi-cell embryo.

15. The mammalian embryo of claim 8, wherein the mammalian embryo is a rodent embryo, optionally a mouse embryo.

16. A method comprising
introducing into the mammalian embryo of any one of the preceding claims (a) a donor polynucleotide comprising a sequence of interest flanked by a first cognate Bxb1 attachment site and a second cognate Bxb1 attachment site, and (b) a Bxb1 integrase or a polynucleotide encoding a Bxb1 integrase.

17. A method comprising
introducing into the mammalian embryo of any one of the preceding claims a donor polynucleotide comprising a sequence of interest flanked by a first cognate Bxb1 attachment site and a second cognate Bxb1 attachment site.

18. The method of claim 16 further comprising implanting the mammalian embryo into a pseudopregnant female mammal.

19. The method of claim 18 further comprising collecting from the female mammal a progeny mammal.

20. The method of claim 19 further comprising screening the progeny mammal for presence or absence of the sequence of interest integrated into the genome of the progeny mammal.

21. The method of claim 16, wherein the donor polynucleotide, the Bxb1 integrase, and/or the polynucleotide encoding a Bxb1 integrase is introduced into the mammalian embryo via microinjection.

22. The method of claim 16, wherein the sequence of interest comprises a gene of interest.

23. The method of claim 16, wherein the sequence of interest has a size of at least 10 kb, at least 15 kb, at least 20 kb, at least 25 kb, or at least 30 kb.

24. A method for producing a Bxb1 landing pad mammal, the method comprising:
(a) introducing into a mammalian embryo (i) a Cas9 nuclease or a polynucleotide encoding a Cas9 nuclease, (ii) a first guide RNA (gRNA) or a polynucleotide encoding a gRNA that targets a first genomic site in the mammalian embryo, (iii) a first single-stranded DNA (ssDNA) donor comprising a first Bxb1 attachment site flanked by a left homology arm and a right homology arm; optionally (iv) a second guide RNA (gRNA) or a polynucleotide encoding a gRNA that targets a second genomic in the mammalian embryo, and (v) a second ssDNA comprising a second Bxb1 attachment site flanked by a left homology arm and a right homology arm; and
(b) implanting the mammalian embryo cell into a pseudopregnant female mammal, wherein the pseudopregnant female mammal is capable of giving birth to a progeny mammal.

25. The method of claim 24, wherein the first ssDNA further comprises a second Bxb1 attachment site upstream or downstream from the first Bxb1 attachment site, wherein both the first and second Bxb1 attachment sites are flanked by the left homology arm and the right homology arm.

26. The method of claim 24, wherein the mammalian embryo comprises a polynucleotide encoding a Bxb1 integrase, or step (a) further comprises introducing into the mammalian embryo a polynucleotide encoding a Bxb1 integrase.

27. The method of claim 24 further comprising collecting the progeny mammal.

28. The method of claim 24, wherein the mammalian embryo is a rodent embryo, optionally a mouse embryo.

29. A mammal comprising the mammalian embryo of claim 8.

30. The mammal of claim 29, wherein the mammal is a rodent, optionally a mouse.

31. A mammal comprising within its genome a Bxb1 attachment site.

32. The mammal of claim 31 further comprising a polynucleotide encoding a Bxb1 integrase.

33. The mammal of claim 31, wherein the Bxb1 attachment site is an attP site, a modified attP* site, an attB site, or a modified attB* site.

34. The mammal of claim 33, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.

35. The mammal of claim 31, wherein the Bxb1 attachment site is within a safe harbor locus, optionally a Rosa26 locus

36. The mammal of claim 31, wherein the mammal is a rodent, optionally a mouse.

37. A mammalian embryo comprising within its genome a Bxb1 attachment site.

38. The mammalian embryo of claim 37 further comprising a polynucleotide encoding a Bxb1 integrase.

39. The mammalian embryo of claim 37, wherein the Bxb1 attachment site is an attP site, a modified attP* site, an attB site, or a modified attB* site.

40. The mammalian embryo of claim 39, wherein the attP site comprises the sequence of SEQ ID NO: 1, the modified attP* site comprises the sequence of SEQ ID NO: 7, the attB site comprises the sequence of SEQ ID NO: 2, and/or the modified attB* site comprises the sequence of SEQ ID NO: 8.

41. The mammalian embryo of claim 37, wherein the Bxb1 attachment site is within a safe harbor locus, optionally a Rosa26 locus.

42. The mammalian embryo of claim 37, wherein the mammalian embryo is a single-cell embryo or a multi-cell embryo.

43. The mammalian embryo of claim 37, wherein the mammalian embryo is a rodent embryo, optionally a mouse embryo.

44. A method comprising
introducing into the mammalian embryo of any one of claims 37-43 (a) a donor polynucleotide comprising a sequence of interest and a cognate Bxb1 attachment site and (b) a Bxb1 integrase or a polynucleotide encoding a Bxb1 integrase.

45. A method comprising
introducing into the mammalian embryo of any one of claims 37-43 a donor polynucleotide comprising a sequence of interest and a cognate Bxb1 attachment site.

46. The method of claim 44 further comprising implanting the mammalian embryo into a pseudopregnant female mammal.

47. The method of claim 46 further comprising collecting from the female mammal a progeny mammal.

48. The method of claim 47 further comprising screening the progeny mammal for presence or absence of the sequence of interest integrated into the genome of the progeny mammal.

49. The method of claim 44, wherein the donor polynucleotide, the Bxb1 integrase, and/or the polynucleotide encoding a Bxb1 integrase is introduced into the mammalian embryo via microinjection.

50. The method of claim 44, wherein the donor polynucleotide is a minicircle.

51. The method of claim 44, wherein the sequence of interest comprises a gene of interest.

52. The method of claim 44, wherein the sequence of interest has a size of at least 3 kb, at least 4 kb, at least 5 kb, at least 6 kb, at least 7 kb, at least 8 kb, at least 9 kb, or at least 10 kb.

53. A method for producing a Bxb1 landing pad mammal, the method comprising:
(a) introducing into a mammalian embryo
(i) a Cas9 nuclease or a polynucleotide encoding a Cas9 nuclease,
(ii) a guide RNA (gRNA) or a polynucleotide encoding a gRNA that targets a genomic locus in the mammalian embryo, and
(iii) a single-stranded DNA (ssDNA) donor comprising a Bxb1 attachment site flanked by a left homology arm and a right homology arm; and
(b) implanting the mammalian embryo cell into a pseudopregnant female mammal, wherein the pseudopregnant female mammal is capable of giving birth to a progeny mammal.

54. The method of claim 53, wherein the mammalian embryo comprises a polynucleotide encoding a Bxb1 integrase, or step (a) further comprises introducing into the mammalian embryo a polynucleotide encoding a Bxb1 integrase.

55. The method of claim 54 further comprising collecting the progeny mammal.

56. The method of claim 55, wherein the mammalian embryo is a rodent embryo, optionally a mouse embryo.

57. A mammal comprising the mammalian embryo of claim 37.

58. The mammal of claim 57, wherein the mammal is a rodent, optionally a mouse.
